(19) United States
(12) Patent Application Publication
(10) Pub. No.: US 2026/0041312 A1
(43) Pub. Date: Feb. 12, 2026

(54) ENDOSCOPIC DEVICE FOR MONITORING SLEEP-RELATED BREATHING

(71) Applicants: YEN-TSUNG LIN, Taipei City (TW); KUANG-CHAO CHEN, New Taipei City (TW)

(72) Inventors: YEN-TSUNG LIN, Taipei City (TW); KUANG-CHAO CHEN, New Taipei City (TW)

(21) Appl. No.: 19/284,708

(22) Filed: Jul. 30, 2025

(30) Foreign Application Priority Data
Aug. 9, 2024 (TW) 113129992

(51) Int. Cl. A61B 1/01; A61B 1/00; A61B 1/233; A61B 5/00

(52) U.S. Cl. CPC A61B 1/01; A61B 1/00016; A61B 1/00114; A61B 1/00137; A61B 1/233; A61B 5/4818

(57) ABSTRACT

The present invention is an endoscopic device for monitoring sleep-related breathing. It includes a main body, an endoscopic transmission cable, an angle-assisting member, an endoscopic lens, and a protective end cap. The endoscopic transmission cable includes a transmission end portion, a signal conduit portion, and a protective base shell. The endoscopic lens is installed in a receiving chamber of the protective base shell and electrically connected to the signal conduit portion. The protective end cap is directly molded and bonded with transparent adhesive onto one end of the protective base shell. The angle-assisting member is mounted on the signal conduit portion near the protective base shell and can adjust and restrict the signal conduit portion to form a seventy-degree angle. Accordingly, the user can insert the lens-equipped end of the endoscopic transmission cable into the nasal cavity and accurately position it at the target area without shifting.

### FIELD OF THE INVENTION

The present invention relates to an endoscopic device for monitoring sleep-related breathing and, more particularly, to a technology applicable in the field of sleep monitoring. Specifically, the present invention aims to enable precise alignment of the endoscope with the target imaging area after insertion deep into the nasal cavity and to reduce issues such as shifting. It also concerns the assembly configuration between the endoscopic lens and the end cap, which is designed to prevent lens instability or detachment within the nasal cavity.

### DESCRIPTION OF RELATED ART

The so-called endoscope refers to a medical instrument used to observe internal areas of the body by being inserted through various passages. The current discussion concerns an endoscopic instrument designed for the detection of sleep apnea. Commercially available systems provide users with a related instrument, which mainly consists of a host unit capable of detection and recording and an endoscopic transmission cable designed to be attached to the human body and inserted into the nasal cavity. One end of the endoscopic transmission cable connects to the host unit, while the other end contains the lens and must be inserted deep into the nasal cavity. After being inserted to a certain length, the lens is then adjusted to the desired imaging position, after which detection can commence.

However, since users are not professional medical personnel and are unfamiliar with the adjustment or operation of the endoscopic transmission cable, misalignment of the lens often occurs, which can cause discomfort inside the nasal cavity or even injury to its interior. Furthermore, to protect the lens from dust, dirt, scratches, and other potential damage, as well as to prevent contact with bodily fluids during use, a protective cap is installed at the end of the endoscopic transmission cable where the lens is located. In traditional endoscope assemblies, a base shell is typically provided at one end of the endoscopic transmission cable, with the base shell recessed to create a receiving space. An additional detachable end cap is attached to the base shell, with the lens installed inside the receiving space. The detachable end cap is transparent, allowing the lens to capture external images. However, the detachable end cap must be molded separately, which increases both inconvenience and cost. Additionally, the assembly process is time-consuming and labor-intensive, prone to misalignment, and carries the risk of the lens being accidentally touched during manual assembly—potentially causing damage and leading to reduced or lost endoscope functionality.

### SUMMARY OF THE INVENTION

The primary objective of the present invention is to enable users to position the end of the endoscopic transmission cable containing the lens at the desired imaging location. Additionally, it provides a cost-effective solution for protecting the lens during installation. This represents an improvement over conventional techniques used in endoscopic transmission cables for detecting sleep apnea, which do not effectively allow users to position the lens accurately. Moreover, the traditional combination of the base shell and the end cap for mounting the lens is costly, time-consuming, labor-intensive, and prone to causing lens misalignment.

To achieve the above-mentioned effects and overcome the shortcomings of conventional techniques, the present invention provides an endoscopic device for monitoring sleep-related breathing, which comprises: a main body; an endoscopic transmission cable including a transmission end portion, a signal conduit portion, and a protective base shell, wherein the transmission end portion is detachably inserted into a connecting part of the main body and electrically connected to the signal conduit portion, and the protective base shell is recessed inward at its end surface to form a receiving chamber; an angle-assisting member installed on the signal conduit portion near the protective base shell and configured to adjust and restrict the signal conduit portion to form an angle of seventy degrees; an endoscopic lens installed in the receiving chamber and electrically connected to the signal conduit portion; and a protective end cap combined with the protective base shell by being directly molded using transparent adhesive after the endoscopic lens is installed in the receiving chamber. The angle-assisting member bends and fixes the signal conduit portion to form a seventy-degree angle, enabling the user to insert the end of the endoscopic transmission cable containing the endoscopic lens into the nasal cavity and accurately position it at the desired imaging location without shaking. The protective end cap is directly molded with transparent adhesive onto one end of the protective base shell to protect the installation of the endoscopic lens.

### BRIEF DESCRIPTION OF THE DRAWINGS

FIG. 1 is a perspective schematic view of the present invention.

FIG. 2 is a partially enlarged view of FIG. 1.

FIG. 3 is a block diagram of the present invention.

FIG. 4 is a schematic view illustrating the molding of the protective end cap of the present invention.

FIG. 5 is a schematic view illustrating the use state of the present invention when installed on the user's face.

FIG. 6 is a schematic view, corresponding to FIG. 5, illustrating the use state of the present invention when the endoscopic cable is inserted into the nasal cavity.

### DETAILED DESCRIPTION OF THE PREFERRED EMBODIMENTS

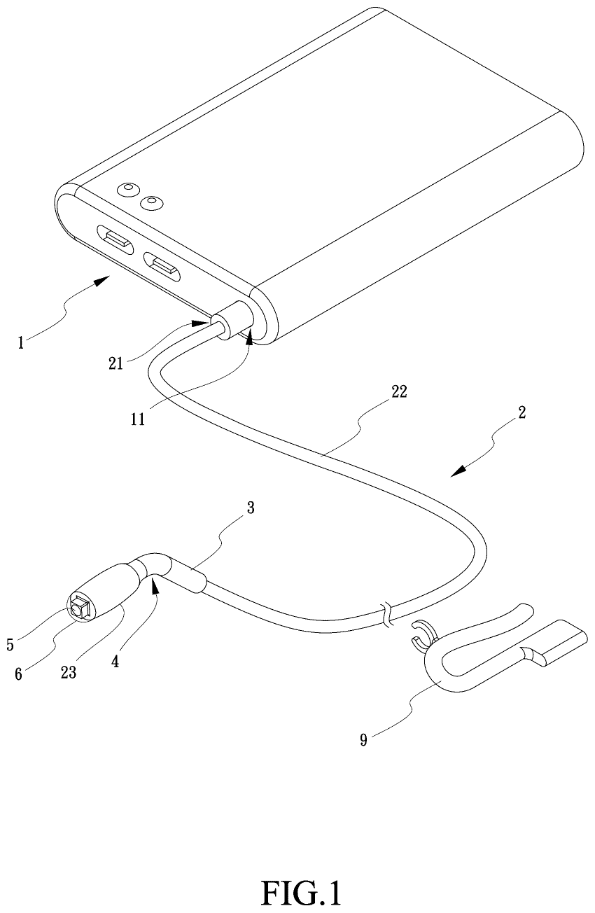

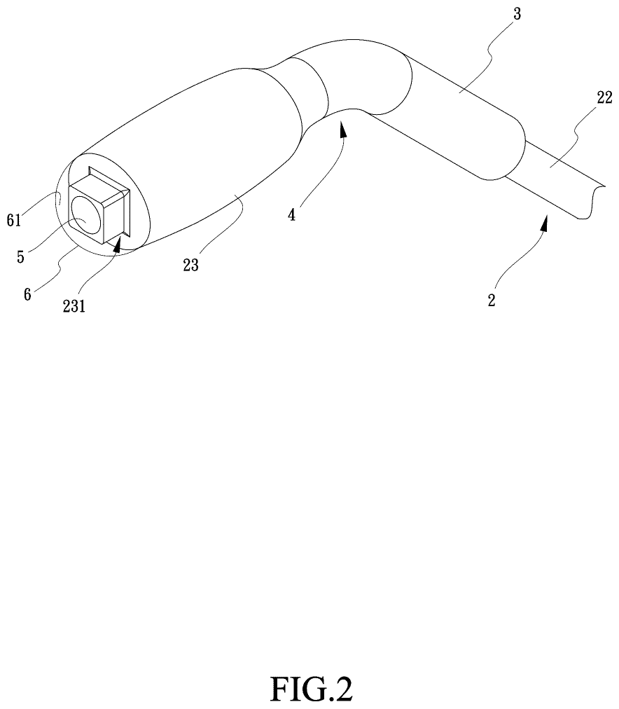

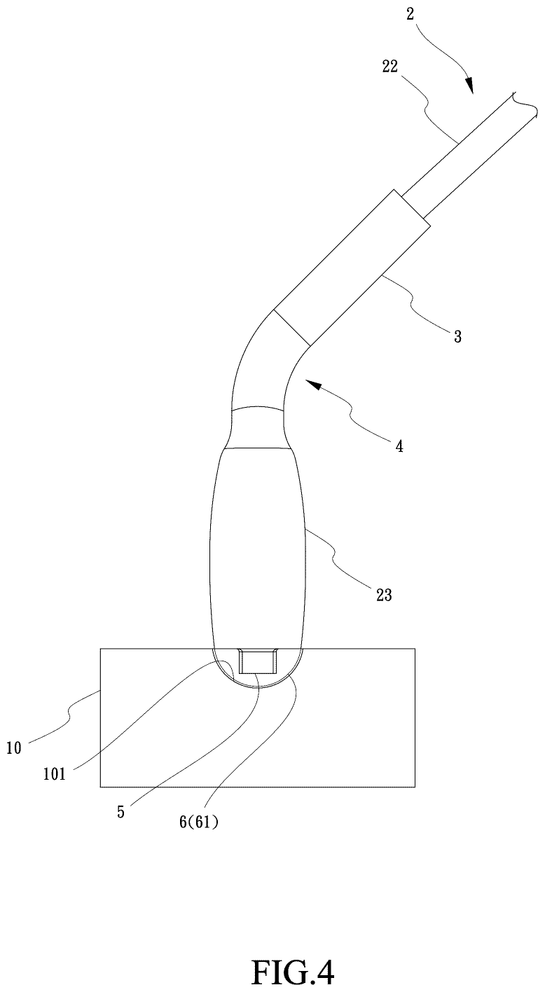

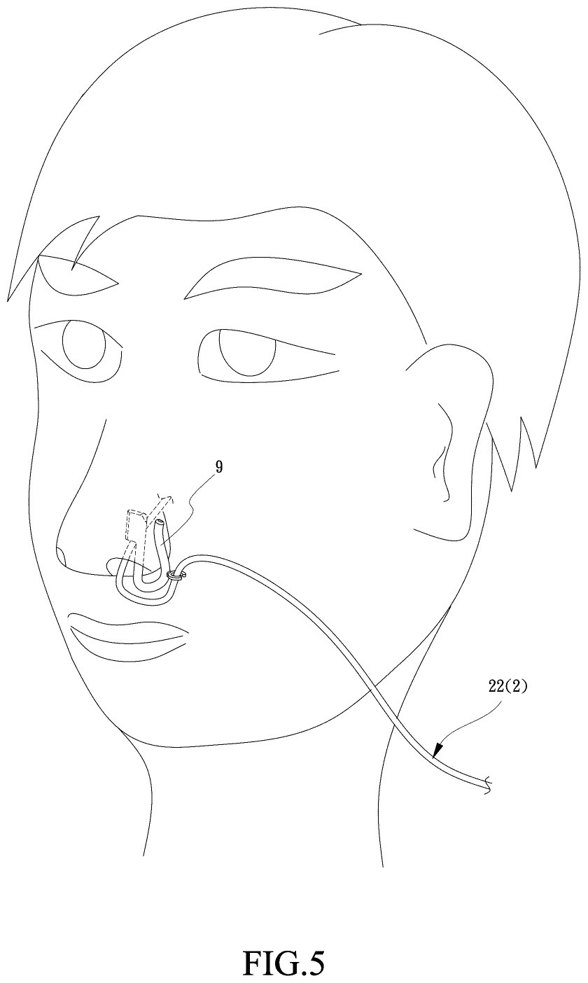

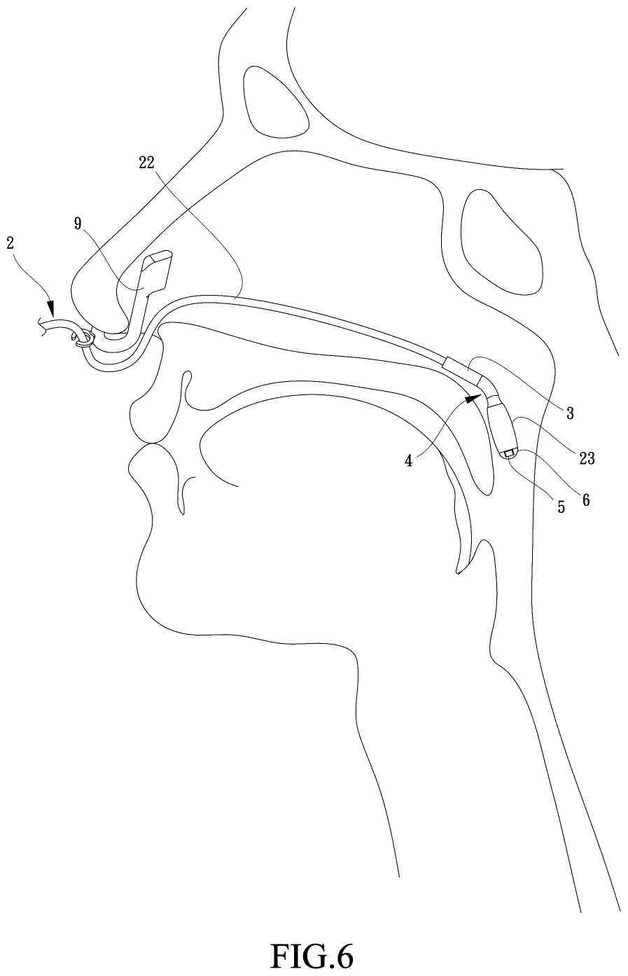

Please refer to FIGS. 1 to 6. The present invention provides an endoscopic device for monitoring sleep-related breathing, mainly applied to the human body, as shown in FIGS. 5 and 6, to monitor the occurrence and duration of sleep apnea caused by the relaxation of soft tissues near the throat during sleep, which in turn leads to upper airway obstruction and narrowing. It mainly includes: a main body 1; an endoscopic transmission cable 2, which includes a transmission end portion 21, a signal conduit portion 22, and a protective base shell 23, the transmission end portion 21 being detachably inserted into a connecting part 11 of the main body 1 and electrically connected to the signal conduit portion 22, and the protective base shell 23 being recessed inward at its end surface to form a receiving chamber 231; an angle-assisting member 3, which is installed on the signal conduit portion 22 near the protective base shell 23 and is configured to adjust and restrict the signal conduit portion 22 to form an angle 4 of seventy degrees; an endoscopic lens 5, installed in the receiving chamber 231 and electrically connected to the signal conduit portion 22; and a protective end cap 6, which is directly molded and bonded to the protective base shell 23 using transparent adhesive after the endoscopic lens 5 is installed in the receiving chamber 231. By using the angle-assisting member 3 to bend and fix the signal conduit portion 22 to form the angle 4 of seventy degrees, the user can insert the end of the endoscopic transmission cable 2, along with the endoscopic lens 5, into the nasal cavity, allowing it to be positioned at the desired imaging location without shaking. The protective end cap 6 is directly molded with transparent adhesive onto one end of the protective base shell 23 to protect the installation of the endoscopic lens 5.

According to the aforementioned technical features defined by the present invention, the main technical points can be divided into two aspects. The first aspect concerns the protective end cap 6. To solve the limitations of conventional methods—where the detachable end cap and the base shell are manufactured and assembled as separate components, requiring an additional high-cost mold for the detachable end cap and resulting in cost inefficiency—the present invention employs transparent adhesive and direct molding to combine and secure the protective end cap 6 with the protective base shell 23. This approach avoids issues associated with the conventional assembly process that may affect the endoscopic lens, reduces costs by eliminating the need for a separate mold for the detachable end cap through direct molding of the protective end cap 6, and saves labor and production steps. The second aspect concerns the endoscopic transmission cable, which in conventional designs is typically a standard electrical wire that cannot be positioned at a specific angle. As a result, the endoscope lens cannot be fixed at the desired imaging position within the nasal cavity—especially since its position may shift when the user turns during sleep. To obtain the most accurate monitoring data, the angle-assisting member 3 fixes the bent angle of the signal conduit portion 22. The user only needs to slightly rotate or adjust the angle of the signal conduit portion 22 inserted into the nasal cavity. Once the protective base shell 23 containing the endoscopic lens 5 reaches the predetermined position, the lens 5 remains fixed regardless of the user's movements, thereby improving monitoring accuracy.

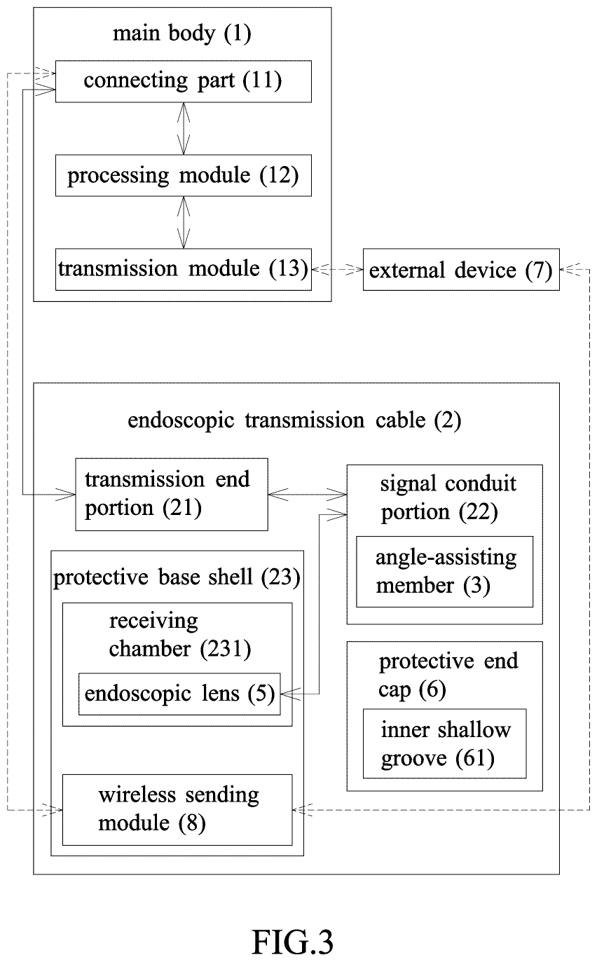

In addition to the main features described above, further technical details and structural elements of the present invention are described. Please refer to FIGS. 1 and 3. To effectively monitor the user's breathing status during sleep, the main body 1 further includes a processing module 12 and a transmission module 13 inside. The endoscopic transmission cable 2, with the end containing the endoscopic lens 5, is inserted into the nasal cavity to capture images. The captured content is transmitted through the signal conduit portion 22 to the transmission end portion 21, which sends the information to the transmission module 13 within the main body 1. The transmission module 13 delivers the received information to the processing module 12 for recording, computation, and storage. In addition, the main body 1 can transmit the received information externally via the transmission module 13 to an external device 7. The external device 7 can be any of a terminal device, mobile phone, tablet and server. The terminal device, which may be located at clinics or at the development and manufacturing site of the main body 1, is used to collect user data during operation. The mobile phone or tablet may belong to the user or their relatives, enabling real-time information reception and immediate emergency handling. The server functions similarly to the terminal device but is primarily intended for sales or clinical use, enabling monitoring information to be uploaded for immediate review and processing. The above examples are illustrative only and not intended to limit the types of external devices 7.

In addition, to adapt to the development of current technology, the present invention may also utilize a direct transmission mold, enabling a connection to the main body 1 without routing through the signal conduit portion 22 and the transmission end portion 21. Therefore, the receiving chamber inside the protective base shell 23 further includes a built-in wireless sending module 8. The wireless sending module 8 wirelessly connects to the transmission module 13 within the main body 1. Thus, the present invention allows for two selectable transmission paths to send images, detection results, and data captured by the endoscopic lens 5 to the main body 1. The first path requires the signal conduit portion 22 and the transmission end portion 21 for connection to the main body 1. The other path allows the wireless sending module 8 to pair directly with the transmission module 13 inside the main body 1. In this way, data can be transmitted directly. Similarly, the wireless sending module 8 can also be paired and connected with the aforementioned external device 7, allowing data to be transmitted directly. This enables users, relatives, and attending medical personnel to monitor the user's breathing condition during sleep in real time, thereby facilitating the most appropriate treatment.

Furthermore, to secure the installation of the device provided by the present invention on the human body, a clip 9 is further installed between the angle-assisting member 3 and the transmission end portion 21. The clip 9 clamps onto the nasal wing after the signal conduit portion 22 of the endoscopic transmission cable 2 is inserted into the nasal cavity, thereby preventing detachment during sleep that would interfere with monitoring.

Finally, regarding the design of the protective end cap 6, to address the drawback in conventional methods that require a separate mold for manufacturing, the present invention adopts an improved approach. Specifically, the protective base shell 23, which houses the endoscopic lens 5, is placed upside down into a mold 10 that features a half-arc mold cavity 101. The base shell 23 is first positioned at the opening of the half-arc mold cavity 101. Then, transparent adhesive is injected into the half-arc mold cavity 101, filling the space within the half-arc mold cavity 101. The transparent adhesive adheres to the protective base shell 23. After air drying and hardening, it forms the protective end cap 6. The end of the protective end cap 6 facing the receiving chamber 231 forms an inner shallow groove 61, which is connected to the receiving chamber 231. This design is primarily to avoid adhesion to the endoscopic lens 5 that would impair imaging and monitoring functions. The depth and range of the inner shallow groove 61 are designed to be just sufficient to avoid any contact with the endoscopic lens 5.

What is claimed is:

1. An endoscopic device for monitoring sleep-related breathing, the device comprising:
a main body;
an endoscopic transmission cable, including a transmission end portion, a signal conduit portion, and a protective base shell, wherein the transmission end portion is detachably inserted into a connecting part of the main body and electrically connected to the signal conduit portion, and the protective base shell is recessed inward at an end surface thereof to form a receiving chamber;
an angle-assisting member, installed on the signal conduit portion near the protective base shell and configured to adjust and restrict the signal conduit portion to form an angle of seventy degrees;
an endoscopic lens, installed in the receiving chamber and electrically connected to the signal conduit portion; and
a protective end cap,
wherein the angle-assisting member bends and fixes the signal conduit portion to form the angle of seventy degrees, enabling a user to insert an end of the endoscopic transmission cable containing the endoscopic lens into a nasal cavity and position the endoscopic lens at a desired imaging location without shaking, and the protective end cap is directly molded with transparent adhesive onto an end of the protective base shell to protect the installation of the endoscopic lens.

2. The endoscopic device for monitoring sleep-related breathing of claim 1, wherein the main body further includes a processing module and a transmission module inside; the endoscopic transmission cable is configured to allow the end containing the endoscopic lens to be inserted into the nasal cavity to capture images and to transmit captured contents through the signal conduit portion to the transmission end portion capable of sending information to the transmission module within the main body; and the transmission module delivers received information to the processing module for recording, computation, and storage.

3. The endoscopic device for monitoring sleep-related breathing of claim 2, wherein the transmission module within the main body is further wirelessly connected to an external device, and the external device is anyone of a terminal device, mobile phone, tablet and server.

4. The endoscopic device for monitoring sleep-related breathing of claim 2, wherein a built-in wireless sending module is further included in the receiving chamber inside the protective base shell and wirelessly connected to the transmission module within the main body.

5. The endoscopic device for monitoring sleep-related breathing of claim 1, wherein a clip is disposed between the angle-assisting member and the transmission end portion for clamping onto a nasal wing after the signal conduit portion of the endoscopic transmission cable is inserted into the nasal cavity.

6. The endoscopic device for monitoring sleep-related breathing of claim 1, wherein an end of the protective end cap faces the receiving chamber and is recessed inward to form an inner shallow groove that connects with the receiving chamber to enclose the endoscopic lens.

7. The endoscopic device for monitoring sleep-related breathing of claim 1, wherein the protective end cap is formed by placing the end of the protective base shell, which is recessed to define the receiving chamber, into a half-arc mold cavity, followed by pouring the transparent adhesive into a space between the half-arc mold cavity and the protective base shell; and the transparent adhesive conforms to a shape of the half-arc mold cavity and, after air drying and hardening, forms the complete protective end cap fixedly bonded to the protective base shell.
